(19) United States
(12) Patent Application Publication
(10) Pub. No.: US 2021/0247342 A1
(43) Pub. Date: Aug. 12, 2021

(54) SYSTEMS AND METHODS FOR AN AIR QUALITY MONITOR FOR DETECTING MULTIPLE LOW CONCENTRATION GAS LEVELS AND PARTICULATE MATTER

(71) Applicant: AerNos, Inc., San Diego, CA (US)

(72) Inventors: Sundip R. DOSHI, San Diego, CA (US); Moazzem HOSSAIN, San Jose, CA (US); Herve LAMBERT, Coronado, CA (US); Albert CHEN, Poway, CA (US)

(21) Appl. No.: 17/242,160

(22) Filed: Apr. 27, 2021

Related U.S. Application Data
(63) Continuation-in-part of application No. 16/858,313, filed on Apr. 24, 2020.
Application No. 16/858,313 is a continuation-in-part of application No. 16/547,499, filed on Aug. 21, 2019.
(60) Provisional application No. 62/721,289, filed on Aug. 22, 2018.
(60) Provisional application No. 62/721,293, filed on Aug. 22, 2018.
(60) Provisional application No. 62/721,296, filed on Aug. 22, 2018.
(60) Provisional application No. 62/721,302, filed on Aug. 22, 2018.
(60) Provisional application No. 62/721,306, filed on Aug. 22, 2018.
(60) Provisional application No. 62/721,309, filed on Aug. 22, 2018.
(60) Provisional application No. 62/721,311, filed on Aug. 22, 2018.
(60) Provisional application No. 62/799,466, filed on Jan. 31, 2019.

(51) Int. Cl. G01N 27/12; G01N 33/00; G01N 27/22

(52) U.S. Cl. CPC G01N 27/121; G01N 33/0011; G01N 27/22; G10L 15/22; G01N 27/127; G01N 27/226; G01N 33/0006

(57) ABSTRACT

A device, comprising: an enclosure; a module within the disclosure, the module comprising: a package, the package including: a sensor chip comprising sensor array comprising a plurality of sensing elements, wherein each of the plurality of sensing elements are functionalized with a deposited mixture consisting of hybrid nanostructures and a molecular formulation specifically targeting at least one of a plurality of gases, and wherein each of the plurality of sensing elements comprises a resistance and a capacitance, and wherein at least one resistance and capacitance are altered when the interacting with gaseous chemical compounds, and a mixed signal System on a Chip (SoC), comprising an analog signal conditioning and Analog-to-Digital conversion circuit configured to convert the analog signal into a digital signal, and a low-power processor circuit configured to processes the digital signal using a pattern recognition system implementing gas detection and measurement algorithms; and a particulate matter sensor.

### CROSS-REFERENCE TO RELATED APPLICATIONS

This application is a continuation-in-part of U.S. patent application Ser. No. 16/858,313, filed Apr. 24, 2020, which in turn claims priority as a continuation-in-part to U.S. patent application Ser. No. 16/547,499, filed Aug. 21, 2019, which claims priority to U.S. Provisional Patent Application No. 62/721,289, filed Aug. 22, 2018, U.S. Provisional Patent Application No. 62/721,293, filed Aug. 22, 2018, U.S. Provisional Patent Application No. 62/721,296, filed Aug. 22, 2018, U.S. Provisional Application No. 62/721,302, filed Aug. 22, 2018, U.S. Provisional Patent Application No. 62/721,306, filed Aug. 22, 2018, U.S. Provisional Patent Application No. 62/721,309, filed Aug. 22, 2018, U.S. Provisional Application No. 62/721,311, filed Aug. 22, 2018, U.S. Provisional Patent Application No. 62/799,466, filed Jan. 31, 2019, the contents of which are incorporated herein by reference.

### BACKGROUND

### 1. Technical Field

The embodiments herein relate to air quality monitoring that combines an innovative hybrid nanostructures multi-gas sensor with a more traditional Particulate Matter (PM) sensor into a single connected monitoring device, enabling granular collection of both gas and particulate matter information, providing instant display of critical local air quality data, directly on the device or via another connected device, as well as Cloud-based data storage and data mining, in addition to internet access to a continuously growing health-related database of air quality information and applications.

### 2. Related Art

Short-term effects of exposure to toxic gases on human health may include the following: irritation to the eyes, nose, and throat; upper respiratory infections such as bronchitis and pneumonia; worsening of medical conditions for individuals with asthma, COPD, or emphysema. Research also shows that, even at very low levels, the same gases can have potentially catastrophic impact when exposure occurs for long periods of time. Long-term exposure to air pollution can cause chronic respiratory disease, lung cancer, heart disease, and even damage to the brain, nerves, liver, or kidney.

Sources of toxic gases, such as ground level ozone, nitrogen dioxide, Volatile Organic Compounds (VOCs), or ammonia are present in many human indoor environments. For example, ozone may be generated by certain types of air purifiers, laundry water treatment systems, facial steamers, fruit and vegetable washers, office equipment, vacuum cleaners, refrigerators, or be the result of outdoor ozone finding its way inside by simply opening a window. Ammonia may come from cleaning products, refrigerant, the burning of coal or wood, tobacco smoke, pets, a vehicle idling in the garage, the presence of livestock or fertilizers on the property, certain types of building material, or even the occurrence of a wildfire in the area. Similarly, micro dust (Particulate Matter) may come from a variety of indoor sources such as stoves, heaters, fireplaces, air fresheners, pesticides, tobacco smoke or a HVAC system with poor or deficient filtration.

The World Health Organization (WHO), the US Environmental Protection Agency (EPA) and a multitude of other agencies worldwide have published ample warnings about the impact of indoor and outdoor air pollutants, as well as guidelines on acceptable levels of each pollutant, and ways to report air quality to end users, for example the EPA's Air Quality Index (AQI, https://www.airnow.gov/aqi/aqi-basics/). Companies and organizations are also attempting to capture and formalize related research and initiatives. For instance, the International WELL Building Institute (IWBI™) proposes a standard for buildings and organizations to integrate human health and well-being concerns into the design of living and working spaces (WELL v2, https://v2.wellcertified.com/wellv2/en/air).

Gas sensor technologies have historically not lent themselves well to creating air quality monitoring products that could be deployed in a home or professional indoor environment with the necessary granularity and operational capabilities. Commercially available gas sensors can be cumbersome to use, expensive and have limited performance, e.g. accuracy, selectivity, lowest detection limit, etc. In addition, other major drawbacks may include inability to detect different types of gases at the same time, inability to measure absolute concentration of individual gases, the requirement for frequent re-calibration, a size incompatible with integration into small form factor systems, the reliance on power-hungry techniques such as heating or on technologies not well suited to manufacturing in very high volume.

### SUMMARY

Systems and methods for the accurate data collection of air quality gas information in an indoor environment. The sensing of gases is done by means of a hybrid nanostructure gas sensor array, in conjunction with specialized electronics and algorithms, to selectively identify and measure the concentration of multiple gases at the same time, down to very low levels of concentration—Parts Per Billion (PPB). The gas sensing module can be combined, within the monitoring device, with any good quality, commercially available, PM sensor to provide a complete picture of air quality in the user's environment. The invention also lends itself to usage in an outdoor environment, provided that a more rugged form factor with appropriate weatherproofing is used in the design of the monitoring device.

According to one aspect, a device, comprising: an enclosure; a module within the disclosure, the module comprising: a package, the package including: a sensor chip comprising sensor array comprising a plurality of sensing elements, wherein each of the plurality of sensing elements are functionalized with a deposited mixture consisting of hybrid nanostructures and a molecular formulation specifically targeting at least one of a plurality of gases, and wherein each of the plurality of sensing elements comprises a resistance and a capacitance, and wherein at least one resistance and capacitance are altered when the interacting with gaseous chemical compounds, and a mixed signal System on a Chip (SoC), comprising an analog signal conditioning and Analog-to-Digital conversion circuit configured to convert the analog signal into a digital signal, and a low-power processor circuit configured to processes the digital signal using a pattern recognition system implementing gas detection and measurement algorithms; and a particulate matter sensor.

### BRIEF DESCRIPTION OF DRAWINGS

The patent or application file contains at least one drawing executed in color. Copies of this patent or patent application publication with color drawing(s) will be provided by the Office upon request and payment of the necessary fee.

These and other features, aspects, and embodiments are described below in the section entitled “Detailed Description.”

FIG. 1 illustrates the basic principles to construct a gas sensor;

FIG. 2 is a prospective view of a physical implementation of a hybrid nanostructure gas sensing element in accordance with one embodiment;

FIG. 3 is a diagram illustrating an embodiment of a gas sensor array that can be included in the hybrid nanostructure gas sensing element of FIG. 2;

FIG. 4 is a block diagram of the hybrid nanostructure gas sensor system that incorporates the hybrid nanostructure gas sensing element of FIG. 2 in accordance with one embodiment;

FIG. 5 is a chart showing the flow of gas information through the hybrid nanostructure gas sensor system of FIG. 4;

FIG. 6 is an is an example of IoT module integrating the air quality monitor system of FIG. 8 according to one embodiment;

FIG. 7 is a block diagram illustrating an example wired or wireless system that can be used in connection with various embodiments described herein;

FIG. 8 is a functional block diagram of an example embodiment of an air quality monitor system that can be an implementation of the system illustrated in FIG. 7 and that can be included in the module of FIG. 6;

FIG. 9 is a conceptual view of an example embodiment of an air quality monitor device that can include the module of FIG. 6;

FIG. 10 is a cross-section of the device of FIG. 9; and

FIG. 11 is a depiction of the air flow through the device of FIG. 9, showing the relative positions of PM sensor, gas sensor and other key components.

### DETAILED DESCRIPTION

Embodiments for a hybrid nanostructure gas sensing system are described herein. The disclosure and the various features and advantageous details thereof are explained more fully with reference to the non-limiting embodiments and examples that are described and/or illustrated in the accompanying drawings and detailed in the following. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings.

The architecture embodied in the hybrid nanostructure gas sensing system described herein achieves the basic requirement of selectively identifying the presence of a gas analyte in diverse mixtures of ambient air but it is also designed to identify multiple gases at the same time, to be compatible in terms of size and power with very small form factors (including for mobile and wearable applications), to be easy to Integrate in IoT applications and to be self-calibrating, thus unshackling the application and/or the service provider from the burden and expense of regular re-calibration.

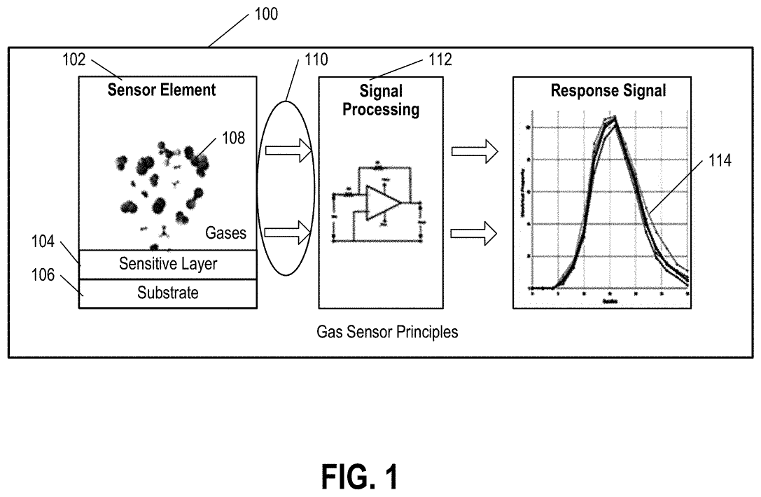

FIG. 1 describes the components of gas sensor 100. As can be seen, such a sensor includes a sensing element 102 that is created by depositing a sensitive layer 104 over a substrate 106. The sensing element 102 can then interact with gaseous chemical compounds 108 altering one or more electrical properties of the sensing element 102. The change in electrical properties can be detected by feeding the sensor raw signals 110 through specially designed signal processing electronics 112. The resulting response signals 114 can be measured and quantified directly or through the application of pattern recognition techniques.

The embodiments described herein comprise six basic elements. The first is the basic sensor element or sensing channel, which combines a structural component, built on a substrate suitable for reliable high-volume manufacturing (some examples described below), with a deposited electrolyte containing hybrid nano structures in suspension. The formulation of the electrolyte is specific to a particular gas or family of gases. A silicon substrate 106 and the structural component can be built using a MEMS manufacturing process. The structural component is essentially an unfinished electrical circuit between two electrodes. The deposition of the electrolyte completes the electrical circuit and, when biased and exposed to gas analytes, changes to one or more of the electrical characteristics of the circuit are used to detect and measure gases.

The second element is the arrangement of multiple sensing channels into an array structure specifically designed and optimized to interface with data acquisition electronics 112. The array structure, combined with the use of pattern recognition algorithms, makes it possible to detect multiple gases at the same time with a single sensor by customizing one or more of the individual sensing channels in the array for a specific gas or family of gases while using other sensing channels to facilitate such critical functions as selectivity.

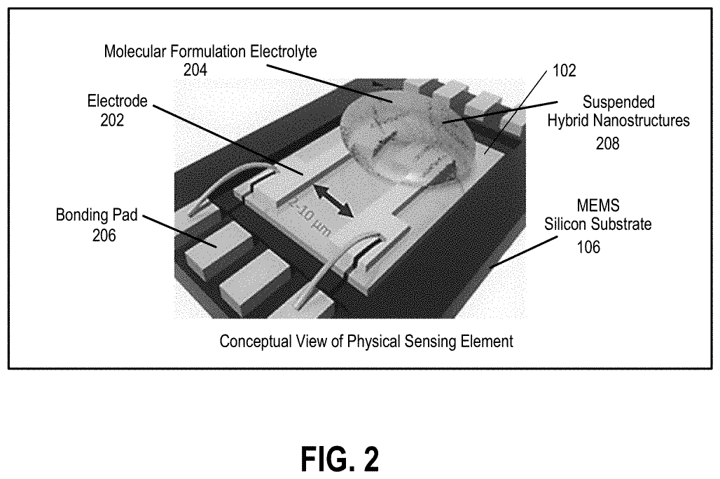

FIG. 2 is a conceptual view of a hybrid nanostructure physical sensing element 102 in accordance with one example embodiment. Different materials can be used for the substrate 106 on which the rest of the sensing element 102 is constructed. But from the perspective of very high volume manufacturing, silicon technology can be preferred and specifically MEMS technology, which provides the necessary foundation for a customer-defined set of manufacturing steps with the flexibility to modulate the complexity of the process based on the sophistication of the sensor chip being built, e.g., to support further innovation or to address special product needs. Silicon technology also provides access to time-proven test methods and multiple sources of Automated Test Equipment that can be customized to fit the needs of gas sensing technology.

The sensing element 102 is made of an incomplete or “open” electrical circuit between two electrodes 202, which is then completed or “closed” by depositing, a molecular formulation electrolyte 204 with hybrid nanostructures 208 in suspension. The process is compatible with several commonly used deposition techniques but does require specially customized equipment and proprietary techniques to achieve the desired quality and reproducibility in a high-volume manufacturing environment. In certain embodiments, the sensing element 102 can be specially patterned to support efficient deposition of nanomaterial in pico-litter amounts and to facilitate incorporation of multiple elements into an array to enables the design of multi-gas sensors.

Electrodes 202 can then be bonded to bonding pads 206 in order to communicate signals 110 to the rest of the system.

One or more molecular formulations may be necessary to completely and selectively identify a particular gas. Combining multiple sensing elements 102, each capable of being “programmed” with a unique formulation, into a sensor array provides the flexibility necessary to detect and measure multiple gases at the same time. It also enables rich functional options such as for instance measuring humidity, an important factor to be accounted for in any gas sensor design, directly on the sensor chip (after all water vapor is just another gas). Another example is the combination for the same gas or family of gases of a formulation capable of very fast reaction to the presence of the gas while another formulation, slower acting, may be used for accurate concentration measurement; this would be important in applications where a very fast warning to the presence of a dangerous substance is required but actual accurate concentration measurement may not be needed at the same time (e.g. first responders in an industrial emergency situation).

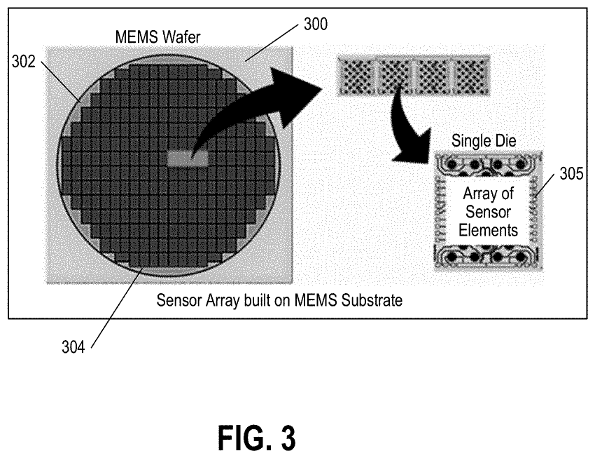

FIG. 3 illustrates the preferred embodiment of a multichannel, gas sensor array 305 where a silicon substrate 302 is used with a MEMS manufacturing process to build the structure of the sensing channels on which the molecular formulations 204 can be deposited. For illustration purposes the size of the individual sensor die 304 is shown as being much larger than achievable in practice; a single 8″ wafer 300 will typically yield several thousand multi-gas capable sensor chips. An array 305 of sensing elements 102 is implemented on a single die 304 and each wafer 300 yields several thousand dies, or chips 304. Each sensing element 102 can then be functionalized by depositing a specific molecular formulation 204 thereon.

Thus, after MEMS manufacturing, additional steps are required to complete the fabrication of each sensing element 102. First, molecular formulations 204 are deposited and cured using specialized equipment. This happens at wafer level and the equipment is designed in a modular fashion to allow for the scaling of the output of a manufacturing facility by duplicating modules and fabrication processes in a copy-exactly fashion. After completion of the manufacturing steps, the wafers 300 must be singulated using a clean dicing technology in order to prevent damage to the sensing elements 102. An example of such technology is Stealth dicing.

The third element is the electronic transducer that detects changes in the electrical characteristics of the sensor array 305, provides signal conditioning and converts the analog signal from the sensor elements 102 into a digital form usable by the data acquisition system, described in more detail below. As described below, the transducer can be a low voltage analog circuit that provides biasing to the array of sensing channels and two functional modes: parking and measurement. Sensing channels are in parking mode either when not in measurement mode or when not used/enabled at all for a given application. The circuitry can be designed to maintain the sensing channels in a linear region of operation, to optimize power consumption, to enable any combination of channels in either parking or measurement modes and to provide a seamless transition between modes.

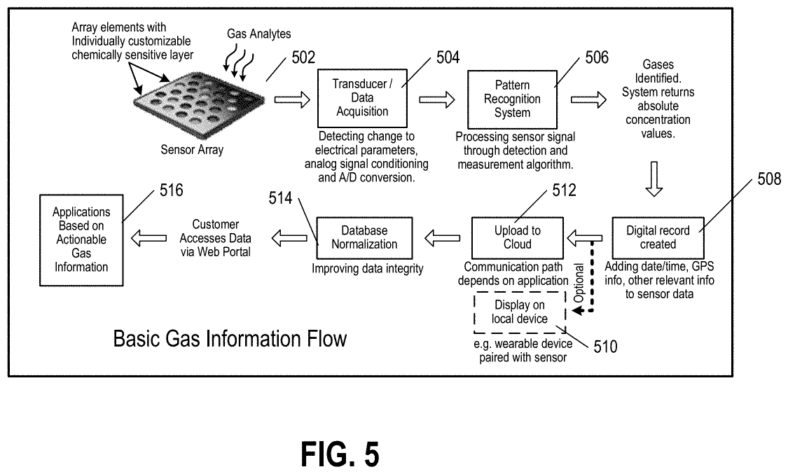

FIG. 5 shows the basic flow of information through a complete nano gas sensor system, such as system 400 described in more detail below. When the sensor array 305 is exposed to the mixture of gas analytes 108 in its environment, in step 502, the sensitive layers 104 of the materials deposited on the sensor elements 102, or sensing channels react, according to their formulation 202, to the presence of specific component gases in the mixture. The reaction causes a change in the electrical characteristics of the sensing channels 102, which is captured by the transducer in the electronics sub-system, in step 504, and then analyzed by the pattern recognition system programmed in the sub-system MCU, in step 506. The output is an absolute value of the concentration of the gases being detected. This is then combined, in step 508, with other desirable meta-data such as time or geo-location into a digital record. This digital record (or a portion of it) can optionally be displayed locally in step 510 (for example, in the case of an application where the sensor is paired to a phone, the data can be further manipulated and displayed by a specially written mobile application running on the phone). More importantly the data is uploaded, via a mechanism that is dependent on the application, to a Cloud data platform in step 512, where the data can be normalized in step 514 and accessed via various application in step 516.

The fourth element is a MCU-based data acquisition and measurement engine, which also provides additional functions such as overall sensor system management and communication, as necessary with encryption, to and from a larger system into which the sensor is embedded.

The third and fourth elements are designed to work together and to form a complete electronic sub-system specifically tuned to work with the array of sensing channels 305 implemented as a separate component. The transducer 404 is firmware configurable to provide optimal A/D conversion for a pattern recognition system running on the MCU 406 and implementing the gas detection and measurement algorithm(s).

The electronic sub-system 402 is suitable for implementation in a variety of technologies depending on target use model and technical/cost trade-offs. PCB implementations will enable quick turn-around and the declination of a family of related products (for instance with different communication interfaces) to support multiple form factors and applications with the same core electronics. When size and power/performance trade-offs are critical, the electronic sub-system 402 is implemented as a System On a Chip (SoC), which can then be integrated together with a MEMS chip carrying the array of sensing channels 305 into a System In a Package (SIP).

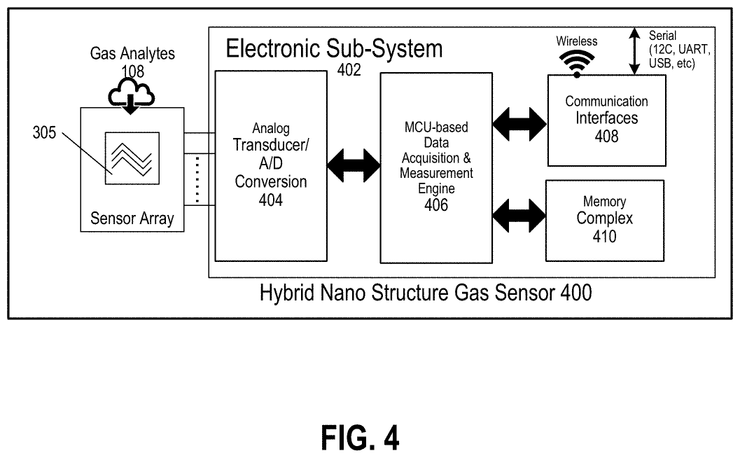

The sensor die 304 must then be assembled with the sensor's electronic sub-system to complete the hybrid nanostructure gas sensor 400 for which a functional block diagram is shown in FIG. 4.

The electronic sub-system can be implemented as a PCB or as a SoC. If the PCB route is followed the sensor die 304 can be either wire-bonded to the electronic sub-system 402 board after completion of the PCB Assembly (PCBA) step or, if the sensor die 304 has itself been individually assembled in a SMT package, it can be soldered on the board as part of PCBA. If the SoC route is followed, the sensor die together with the SoC die of the electronic sub-system 402 can be stacked and assembled together into a single package (System In a Package) or each can possibly be assembled into individual packages.

Either assembled into its own package or assembled into a SIP, the sensor chip 304 must be exposed to ambient air. Therefore, the package lid must include a hole of sufficient size over the sensor.

Testing happens at various points of the sensor manufacturing process.

After sensor functionalization (deposition of the molecular formulations 204), certain handling precautions must be followed for the rest of the product manufacturing flow to prevent accidental damage to the sensor chip 304 (e.g. a pick and place tool must not make contact with the surface of the sensing elements).

The fifth element is the gas detection and measurement algorithm. The algorithm implements a method for predicting target gas concentration by reading the hybrid nanostructure sensor array's multivariate output and processing it inside the algorithm. The algorithm analyzes sensor signals in real time and outputs estimated values for concentrations of target gases. The algorithm development is based on models that are specific to the materials deposited on the sensing channels of the sensor array. These models are trained based on the collection of an abundant volume of data in the laboratory (multiple concentrations of target gases, combinations of gases, various values of temperature, relative humidity and other environmental parameters). Sophisticated supervised modeling techniques are used to attain the best possible agreement between true and predicted values of target gas concentrations. Prior to deployment, extensive lab and field testing is carried out to optimize model performance and finalize sensor validation.

The first five elements together constitute the hybrid nanostructure gas sensor 400 and provide all the functionality necessary to detect multiple gases 108 in ambient air at the same time and to report their absolute concentrations. The sensing capability of the hybrid nanostructure sensor array 305 is always “on” and the gas detection and measurement algorithm makes it possible for the sensor 400 to require no special calibration step before use and to remain self-calibrating through its operational life.

The sixth element is the Cloud Data Platform that enables a virtually unlimited number of sensors 400 deployed as part of a virtually unlimited number of applications to be hosted in a global database where big data techniques can be used to analyze, query and visualize the information to infer actionable insight. The use of a Cloud-based environment provides all the necessary flexibility to customize how the data can be partitioned, organized, protected and accessed based on the rights of individual tenants.

The Cloud data platform provides another layer of sophistication to the system by allowing Cloud applications to operate on the data set. For instance, sensors 400 that are located in the same vicinity would typically report consistent gas values thus allowing errant results to be identified and a possible malfunction of one node of a network of sensors investigated.

The continuous collection of highly granular gas information by a multitude of connected devices (IoT—Internet Of Things) is critical to go beyond monitoring to generate actionable insight from large amount of collected data (Big Data Analytics, Artificial Intelligence).

A few application examples are highlighted below.

Example 1: We take 20,000 breaths every day and the air we breathe impacts our health—the science is already clear on this—but we rarely know what is in the air we breathe. To take meaningful action, consumers, scientists, public officials and business owners need the ability to measure air pollution at a personal, local and granular level which has, previously been impossible due to the limitations of commercially available gas sensors mentioned above.

Mounting evidence suggests that prenatal and early life exposure to common environmental toxins, such as air pollution from fossil fuels, can cause lasting damage to the developing human brain. These effects are especially pronounced in highly vulnerable fetuses, babies, and toddlers as most of the brain's structural and functional architecture is established during these early developmental periods. These disruptions to healthy brain development can cause various cognitive, emotional, and behavioral problems in later infancy and childhood.

The sensor technology described herein allows researchers to gather highly detailed, accurate data about pregnant women's exposure to environmental air pollution and the resulting effects on the developing brain. The availability of this technology will represent a profound advance on current methods and efforts in the field that will have far-reaching consequences for improving newborn and child health throughout the world.

More generally, personal air monitoring and local indoor and outdoor monitoring will be a breakthrough for scientific research, healthcare interventions, personal preventive actions, advocacy and more.

The sensor technology described herein can deliver complete processing and gas results to a broad spectrum of smart systems under development for the Smart Cities of tomorrow. The sensor is designed for Plug and Play integration into IoT devices and the small form factor is compatible with a multitude of devices from LED lights to smart meters, to standalone monitoring stations, to non-stationary devices (drones, public vehicles, wearables, phones, etc.).

Example 2: The sensor technology described herein can be used in smart appliances such as connected refrigerators, that will help customers monitor food freshness, detect spoilage and the presence of harmful pesticide residues. The simultaneous, multi-gas, sensing capability of the invention will enable sensors that can recognize the gas patterns associated with the condition of specific foods.

Example 3: A network or grid of the sensors 400 described herein, can be integrated into industrial areas such as petrochemical complexes and oil refineries to allow companies to monitor the sites during regular operation (e.g. for leaks) or in the event of natural or human-made disasters. The sensors can also be installed in drones for data collection in hard to reach or potentially dangerous area. The ability of the technology to be deployed in wearables and in fixed and mobile networks will provide both personal protection and granular data across large area, allow the constant monitoring of a facility for preventive measures to be taken in a timely fashion, save critical time when urgent decision making is required and provide invaluable information to protect workers and emergency personnel.

The same technology can place powerful new tools in the hands of first responders and officials responsible for public safety and homeland security.

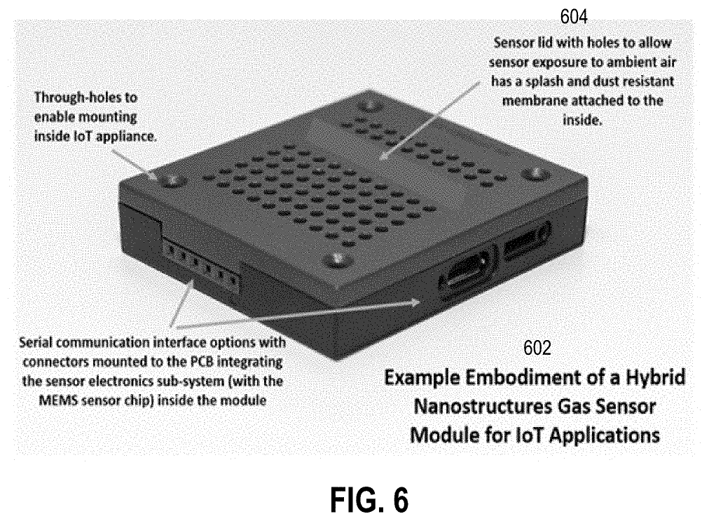

FIG. 6 shows an example embodiment of a hybrid nanostructures gas sensor 602, in this case a module intended for IoT applications. The sensor technology lends itself to integration into any number of IoT devices. While the sensor does not need the active creation of an airflow to function, the sensitive layers at the surface of the sensor must be exposed to ambient air and at the same time provided a reasonable amount of protection from dust and fluids. This can be achieved by designing an air interface that ensures that the sensor is behind a perforated shield, e.g., the lid 604 of an enclosure 606 with a thin membrane (PTFE, 0.5 um mesh) being used to provide splash and dust protection. Outdoor applications can require the design of a more complicated air interface to meet the weather-proofing requirements.

As noted above, the ability to accurately detect multiple gases at the same time, often at parts-per-billion (PPB) sensitivity is becoming crucial to a growing number of industries as well as to the world-wide expansion of air quality monitoring initiatives aiming to address household and urban air pollution challenges. The following outlines in more detail embodiments that combine a nanohybrid gas sensor chip that uses highly sensitive nano-nucleated structures, as described above together with a mixed signals System-On-a-Chip (SoC) in a single, small, and very thin package to deliver the key fundamental attributes required for the broad deployment of sensors capable of low detection limits (PPB) in support of highly granular collection of gas information in ambient air. A hybrid nanostructure gas sensor, as described above, can provide all the functionality necessary to detect multiple gases in ambient air at the same time and to report their absolute concentrations. The sensing capability of the hybrid nanostructure sensor array is always “on”, whereas the gas detection and measurement algorithm enable the sensor to require no special calibration step before use and to remain self-calibrating through its operational life.

The mixed signals SoC, described below, combines highly optimized analog electronics with a microcontroller-based digital backend. The analog portion provides bias to the sensor chip and enables “parking” and measurement” functions for each element of the multi-channel gas sensor array, detects changes in electrical properties of the sensing channels, conditions the raw analog signal from the sensor array, and runs the analog signal through an A/D conversion to provide an input signal to the digital back-end. The digital backend includes a powerful, but very low power microcontroller that provides controls to the analog frontend to optimize power delivery, sensor data collection, and gas concentration measurement. The digital backend runs custom pattern recognition algorithms to calculate and report gas concentration values, manages formatting and temporary accumulation/storage of gas information and other related metadata, and controls communications in and out of the system via a selection of serial interfaces.

Both sensor and SoC chips can be stacked and connected into a state-of-the-art custom-designed package to deliver a complete sensor system solution, a System In a Package (SIP) (described in more detail below), suitable for integration into the most aggressive IoT form factors.

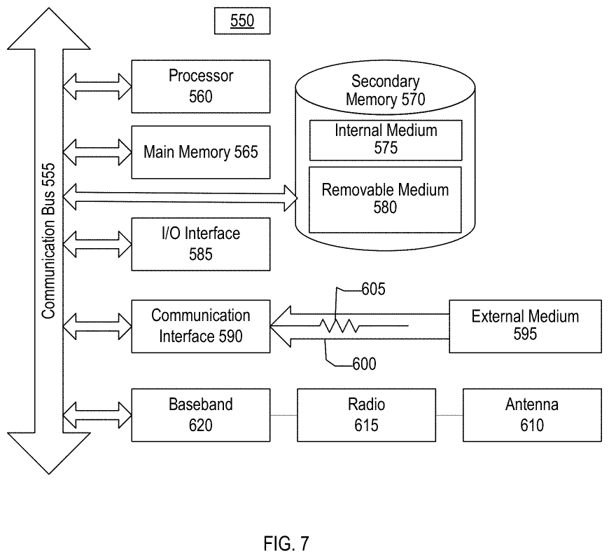

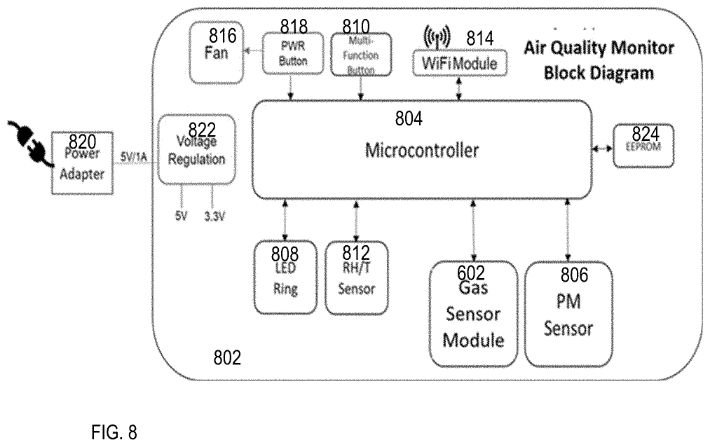

FIG. 8 is a functional block diagram of an example embodiment of an air quality monitor system 802, e.g., and SoC that can be an implementation of the system illustrated in FIG. 7 and that can be included in the module of FIG. 6. The air quality monitoring system can be architected around a microcontroller 804 handling the necessary communication interfaces to the hybrid nanostructures gas sensor 602, a PM sensor 806, a RGB LED ring 808 to provide visual air quality feedback/alert based on a color scheme compliant with, for example, the EPA's Air Quality Index, a multifunction control pad 810, e.g., simple buttons to control power and a limited set of functionality, a RHT sensor 812 to provide ambient humidity and temperature information, and a WiFi module 814, which can also be integrated with the processor 804. System 802 can also include a fan 816 for cooling, a power button 818, a power adapter 820 and voltage regulation circuit 822, as well as memory 824 for storing instructions to be run by processor 804.

Connectivity to the internet ensures that the air quality data from a monitor that incorporates module 602, e.g., as part of a system 802, or as is likely in typical use models, from multiple monitors, e.g. one in every room of a house or professional building, will be uploaded to the Cloud data platform and available to the end user through applications running on other devices connected to the same network, e.g., mobile phone, PC, or other internet appliances. System 802 can also be designed for compatibility with Cloud-based, voice-activated, virtual assistants, such as Google Smart Assistant, Siri or Alexa, or can actually include such capabilities.

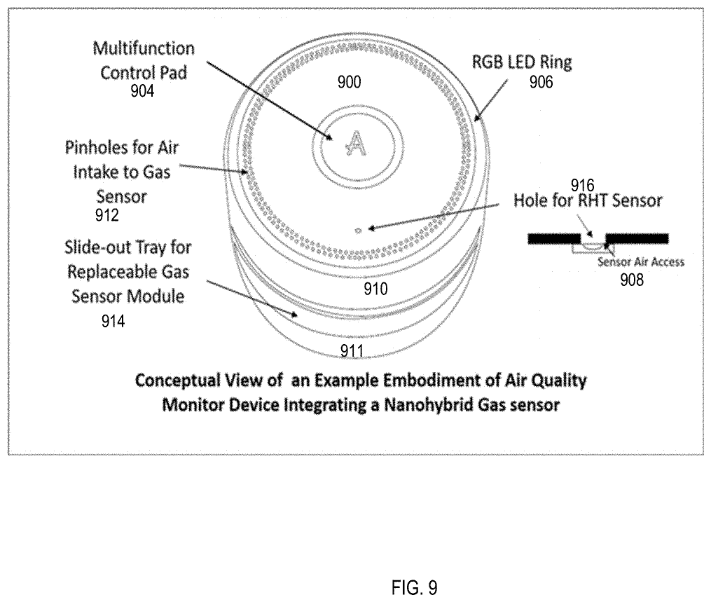

FIG. 9 shows a conceptual rendering of a possible implementation of a device 900 that includes an air quality monitor 802. The power button/multifunction controls 904, LED ring 906 and RHT sensor 908 can all be integrated in the upper portion (lid) 910 of the enclosure 911. The PM sensor (not show), hybrid nanostructures gas sensor module (not shown), and a fan (not shown) must be located in specific sections or chamber of the enclosure 911. The rest of the electronics (not shown), essentially a small PCB including the microcontroller 804, can be located where it makes sense for the physical design of the enclosure 911. An EEPROM device 824 can be added to store information unique to each device 900.

The fan is required to draw ambient air in and out of the enclosure 911. Pinholes 912 in the lid 910 of the enclosure 911 can provide the air intake, while the fan pulls the air through the device 900 down to the gas sensor and out at the bottom of the enclosure 911. Also, lid 910 can include a hole 916 in lid 911 above RHT sensor 908.

In certain embodiments, enclosure 911 can comprise a slide out or removable tray 914 that allows, e.g., module 602 to be inserted and removed.

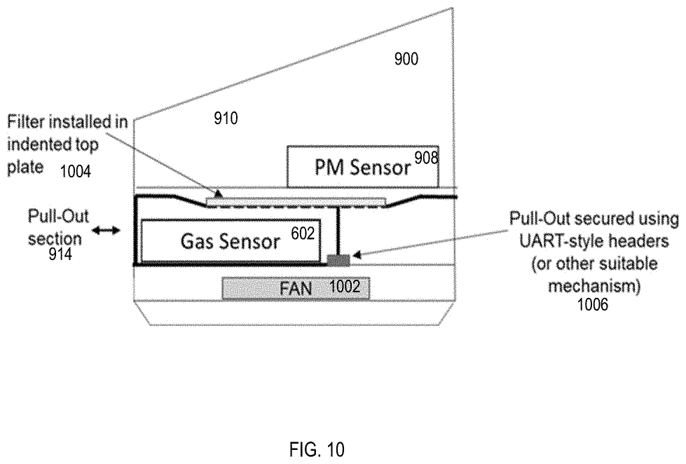

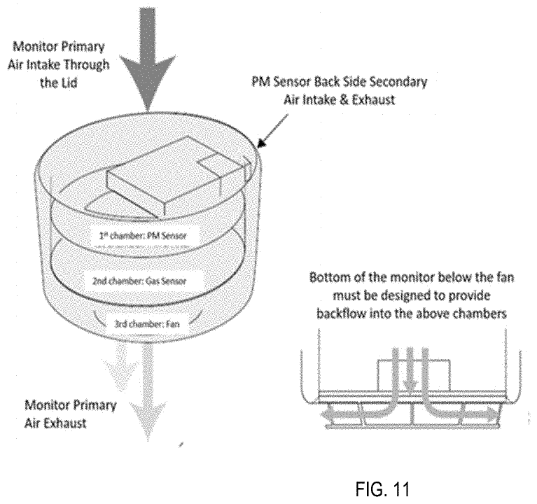

FIG. 10 is a cross-section of device 900. The PM sensor 908 is immediately below the lid 910, while the hybrid nanostructures gas sensor module 602 is on a pull-out tray 914 below it and above the fan 1002. The pull-out tray 914 can be designed such that an optional and replaceable filter 1002 can be installed above the gas sensor module 602 to remove particulate matter. Commercially available PM sensors may differ in the specific mechanical implementation of the sensor enclosure but most often integrate a built-in fan to pull ambient air inside the sensor 908. The enclosure of the air quality monitor must therefore provide a path or guide, e.g., including hole 916, for air in the room to reach the opening in the PM sensor 908 enclosure. In the example of FIGS. 9 and 10 the chosen PM sensor 908 requires additional pinholes on the back of the device 900 and an air guide to direct ambient air to (intake) and from (exhaust) openings in the enclosure 911. FIG. 11 provides an illustration of the air flow through the monitor device 900.

FIG. 7 is a block diagram illustrating an example wired or wireless system 550 that can be used in connection with various embodiments described herein. For example the system 550 can be used as or in conjunction with one or more of the platforms, devices or processes described above, and may represent components of a device, such as sensor 400, the corresponding backend or cloud server(s), and/or other devices described herein. The system 550 can be a server or any conventional personal computer, or any other processor-enabled device that is capable of wired or wireless data communication. Other computer systems and/or architectures may be also used, as will be clear to those skilled in the art.

The system 550 preferably includes one or more processors, such as processor 560. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 560. Examples of processors which may be used with system 550 include, without limitation, the Pentium® processor, Core i7® processor, and Xeon® processor, all of which are available from Intel Corporation of Santa Clara, Calif. Example processor that can be used in system 400 include the ARM family of processors and the new open source RISC-V processor architecture.

The processor 560 is preferably connected to a communication bus 555. The communication bus 555 may include a data channel for facilitating information transfer between storage and other peripheral components of the system 550. The communication bus 555 further may provide a set of signals used for communication with the processor 560, including a data bus, address bus, and control bus (not shown). The communication bus 555 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, or standards promulgated by the Institute of Electrical and Electronics Engineers (IEEE) including IEEE 488 general-purpose interface bus (GPM), IEEE 696/S-100, and the like.

System 550 preferably includes a main memory 565 and may also include a secondary memory 570. The main memory 565 provides storage of instructions and data for programs executing on the processor 560, such as one or more of the functions and/or modules discussed above. It should be understood that programs stored in the memory and executed by processor 560 may be written and/or compiled according to any suitable language, including without limitation C/C++, Java, JavaScript, Pearl, Visual Basic, .NET, and the like. The main memory 565 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

The secondary memory 570 may optionally include an internal memory 575 and/or a removable medium 580, for example a floppy disk drive, a magnetic tape drive, a compact disc (CD) drive, a digital versatile disc (DVD) drive, other optical drive, a flash memory drive, etc. The removable medium 580 is read from and/or written to in a well-known manner. Removable storage medium 580 may be, for example, a floppy disk, magnetic tape, CD, DVD, SD card, etc.

The removable storage medium 580 is a non-transitory computer-readable medium having stored thereon computer executable code (i.e., software) and/or data. The computer software or data stored on the removable storage medium 580 is read into the system 550 for execution by the processor 560.

In alternative embodiments, secondary memory 570 may include other similar means for allowing computer programs or other data or instructions to be loaded into the system 550. Such means may include, for example, an external storage medium 595 and an interface 590. Examples of external storage medium 595 may include an external hard disk drive or an external optical drive, or and external magneto-optical drive.

Other examples of secondary memory 570 may include semiconductor-based memory such as programmable read-only memory (PROM), erasable programmable read-only memory (EPROM), electrically erasable read-only memory (EEPROM), or flash memory (block oriented memory similar to EEPROM). Also included are any other removable storage media 580 and communication interface 590, which allow software and data to be transferred from an external medium 595 to the system 550.

System 550 may include a communication interface 590. The communication interface 590 allows software and data to be transferred between system 550 and external devices (e.g. printers), networks, or information sources. For example, computer software or executable code may be transferred to system 550 from a network server via communication interface 590. Examples of communication interface 590 include a built-in network adapter, network interface card (NIC), Personal Computer Memory Card International Association (PCMCIA) network card, card bus network adapter, wireless network adapter, Universal Serial Bus (USB) network adapter, modem, a network interface card (NIC), a wireless data card, a communications port, an infrared interface, an IEEE 1394 fire-wire, or any other device capable of interfacing system 550 with a network or another computing device.

Communication interface 590 preferably implements industry promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 590 are generally in the form of electrical communication signals 605. These signals 605 are preferably provided to communication interface 590 via a communication channel 600. In one embodiment, the communication channel 600 may be a wired or wireless network, or any variety of other communication links. Communication channel 600 carries signals 605 and can be implemented using a variety of wired or wireless communication means including wire or cable, fiber optics, conventional phone line, cellular phone link, wireless data communication link, radio frequency (“RF”) link, or infrared link, just to name a few.

Computer executable code (i.e., computer programs or software) is stored in the main memory 565 and/or the secondary memory 570. Computer programs can also be received via communication interface 590 and stored in the main memory 565 and/or the secondary memory 570. Such computer programs, when executed, enable the system 550 to perform the various functions of the present invention as previously described.

In this description, the term “computer readable medium” is used to refer to any non-transitory computer readable storage media used to provide computer executable code (e.g., software and computer programs) to the system 550. Examples of these media include main memory 565, secondary memory 570 (including internal memory 575, removable medium 580, and external storage medium 595), and any peripheral device communicatively coupled with communication interface 590 (including a network information server or other network device). These non-transitory computer readable mediums are means for providing executable code, programming instructions, and software to the system 550.

In an embodiment that is implemented using software, the software may be stored on a computer readable medium and loaded into the system 550 by way of removable medium 580, I/O interface 585, or communication interface 590. In such an embodiment, the software is loaded into the system 550 in the form of electrical communication signals 605. The software, when executed by the processor 560, preferably causes the processor 560 to perform the inventive features and functions previously described herein.

In an embodiment, I/O interface 585 provides an interface between one or more components of system 550 and one or more input and/or output devices. Example input devices include, without limitation, keyboards, touch screens or other touch-sensitive devices, biometric sensing devices, computer mice, trackballs, pen-based pointing devices, and the like. Examples of output devices include, without limitation, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), and the like.

The system 550 also includes optional wireless communication components that facilitate wireless communication over a voice and over a data network. The wireless communication components comprise an antenna system 610, a radio system 615 and a baseband system 620. In the system 550, radio frequency (RF) signals are transmitted and received over the air by the antenna system 610 under the management of the radio system 615.

In one embodiment, the antenna system 610 may comprise one or more antennae and one or more multiplexors (not shown) that perform a switching function to provide the antenna system 610 with transmit and receive signal paths. In the receive path, received RF signals can be coupled from a multiplexor to a low noise amplifier (not shown) that amplifies the received RF signal and sends the amplified signal to the radio system 615.

In alternative embodiments, the radio system 615 may comprise one or more radios that are configured to communicate over various frequencies. In one embodiment, the radio system 615 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from the radio system 615 to the baseband system 620.

If the received signal contains audio information, then baseband system 620 decodes the signal and converts it to an analog signal. Then the signal is amplified and sent to a speaker. The baseband system 620 also receives analog audio signals from a microphone. These analog audio signals are converted to digital signals and encoded by the baseband system 620. The baseband system 620 also codes the digital signals for transmission and generates a baseband transmit audio signal that is routed to the modulator portion of the radio system 615. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 610 where the signal is switched to the antenna port for transmission.

The baseband system 620 is also communicatively coupled with the processor 560. The central processing unit 560 has access to data storage areas 565 and 570. The central processing unit 560 is preferably configured to execute instructions (i.e., computer programs or software) that can be stored in the memory 565 or the secondary memory 570. Computer programs can also be received from the baseband processor 610 and stored in the data storage area 565 or in secondary memory 570, or executed upon receipt. Such computer programs, when executed, enable the system 550 to perform the various functions of the present invention as previously described. For example, data storage areas 565 may include various software modules (not shown).

Various embodiments may also be implemented primarily in hardware using, for example, components such as application specific integrated circuits (ASICs), or field programmable gate arrays (FPGAs). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, functions, and methods described in connection with the embodiments disclosed herein can be implemented or performed with a general purpose processor, a digital signal processor (DSP), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such the processor can read information from, and write information to, the storage medium. In the alternative, the storage medium can be integral to the processor. The processor and the storage medium can also reside in an ASIC.

Any of the software components described herein may take a variety of forms. For example, a component may be a stand-alone software package, or it may be a software package incorporated as a “tool” in a larger software product. It may be downloadable from a network, for example, a website, as a stand-alone product or as an add-in package for installation in an existing software application. It may also be available as a client-server software application, as a web-enabled software application, and/or as a mobile application.

While certain embodiments have been described above, it will be understood that the embodiments described are by way of example only. Accordingly, the systems and methods described herein should not be limited based on the described embodiments. Rather, the systems and methods described herein should only be limited in light of the claims that follow when taken in conjunction with the above description and accompanying drawings.

What is claimed is:

1. A device, comprising:
an enclosure;
a module within the disclosure, the module comprising:
a package, the package including:
a sensor chip comprising sensor array comprising a plurality of sensing elements, wherein each of the plurality of sensing elements are functionalized with a deposited mixture consisting of hybrid nanostructures and a molecular formulation specifically targeting at least one of a plurality of gases, and wherein each of the plurality of sensing elements comprises a resistance and a capacitance, and wherein at least one resistance and capacitance are altered when the interacting with gaseous chemical compounds, and a mixed signal System on a Chip (SoC), comprising
an analog signal conditioning and Analog-to-Digital conversion circuit configured to convert the analog signal into a digital signal, and
a low-power processor circuit configured to processes the digital signal using a pattern recognition system implementing gas detection and measurement algorithms; and
a particulate matter sensor.

2. The device of claim 1, further comprising pinholes in the enclosure for air intake to the module.

3. The device of claim 2, further comprising a fan positioned in the enclosure such that is will when operating cause air to flow through the device and bringing air in through the pinholes to the module and then out of the device.

4. The device of claim 1, further comprising a hole and a path within the enclosure to allow air to reach the particulate matter sensor.

5. The device of claim 1 further comprising a multifunction control pad.

6. The device of claim 1, further comprising an LED ring or other indicator configured to indicate the air quality status.

7. The device of claim 1, further comprising a removable portion configured to receive the module.

8. The device of claim 1, further comprising an air filter to filter air as it is presented to the module.

9. The device of claim 1, further comprising communication capability to allow the air quality data to be transmitted to the cloud or other storage platform.

10. The device of claim 1, further comprising communication capability to allow the device to communicate with a virtual assistant or smart speaker.

11. The device of claim 1, further comprising voice recognition and instruction capability.

12. The sensor system in a package of claim 1, wherein the mixed signal SOC further comprises a processor and a memory, coupled with the processor, the memory configured to store algorithms combining models that accurately reflect the behavior of sensing elements customized with the specific molecular formulation, and instruction that cause the processor to perform pattern recognition techniques to convert raw sensor output into gas concentration readings based on the algorithms and models.

13. The sensor system in a package of claim 1, wherein each of the plurality of sensing element is designed such that the hybrid nanostructures and molecular formulations can be deposited using drop casting or electro-chemical deposition.

14. The sensor system in a package of claim 1, wherein each of the plurality of sensing element comprises a MEMS substrate.

15. The sensor system in a package of claim 1, wherein the analog signal conditioning and Analog-to-Digital conversion circuit further comprises a parking circuit and a measurement circuit, wherein the plurality of sensing elements are connected to the parking circuit when not connected to the measurement circuit.

16. The sensor system in a package of claim 5, wherein the parking circuit is further configured to keep the plurality of sensing elements within the linear region of operation, and to effectively switch the plurality of sensing elements between inactive and active modes while reducing the overall power consumption.

17. The sensor system in a package of claim 5, wherein the measurement circuit is configured to minimize settling times when the plurality of sensing elements are being switched between the parking circuit and the measurement circuit.

18. The sensor system in a package of claim 5, wherein the parking and measurement circuits allow for a make before break connection scheme to minimize transient loads on the plurality of sensing elements.

19. The sensor system in a package of claim 5, wherein the parking and measuring circuits comprise switch arrays configured to select and drive up to N sensor elements, which comprise the plurality of sensor elements, a sensor driver, a reference block, a transimpedance amplifier (TIA), a programmable gain amplifier (PGA) configured to take sensor measurement, and an analog to digital converter (ADC) configured to convert each sensor measurement into a digital representation.

20. The sensor system in a package of claim 9, wherein the sensor driver comprises a low offset buffer capable of driving a continuous dc load current and configured to use a reference voltage (Vref) output voltage from the reference block to create a Vref buffered output that is used to force a Vref bias value across all sensor elements of the plurality of sensor elements that are connected to the parking circuit.

21. The sensor system in a package of claim 10, wherein the value of Vref is selected to keep the plurality of sensor elements that are connected with the parking circuit within the linear region of operation, reduce the overall power consumption, and provide a reasonable range of measurement for the digital logic.

22. The sensor system in a package of claim 10, wherein the sensor driver includes an array of N low resistance switches to facilitate connection to the plurality of sensor elements such that each of the plurality of sensor elements can be connected to a driver buffer through a dedicated switch in the switch array.

23. The sensor system in a package of claim 9, wherein the TIA is configured to measure the resistance of a sensor element in the plurality of sensor elements using a calibrated Vref output voltage from the Reference block, and a low offset buffer capable of driving a set minimum current of continuous dc load by forcing a calibrated Vref dc across the sensor element to be measured to measure the sensor resistance.

24. The sensor system in a package of claim 13, wherein the TIA transfer function is given by: VOUT=(1+RFB/RS)VIN, where RFB is the resistance of a TIA feedback resistor and RS is the resistance of the sensor element to be measured, and VIN is the calibrated Vref.

25. The sensor system of claim 14, wherein the TIA generates a pair of differential output signals across the feedback resistor, and wherein the differential output signals are provided to the PGA.

26. The sensor system in a package of claim 14, wherein the feedback resistor can be an external resistor, and internal resistor, or both.

27. The sensor system in a package of claim 16, further comprising two external calibration resistors configured to be provide a precision known resistor instead of a sensor element for calibrating both an unknown sensor element and the feedback resistor in the event an internal resistor is used for the feedback resistor.

28. The sensor system in a package of claim 17, wherein the TIA includes an array of N low resistance switches to facilitate connection to sensor elements of the plurality of sensor elements.

29. The sensor system in a package of claim 18, further comprising additional switches are included to facilitate connection to calibration resistors.

30. The sensor system in a package of claim 9, wherein the PGA output signals are connected to the inputs of the ADC, and wherein the ADC uses a 16-bit second order Sigma Delta converter with 1-bit quantization to generate a digital representation of the PGA output voltage.

31. The sensor system in a package of claim 1, wherein a subset of the plurality of sensor elements are configured to measure humidity.

32. The sensor system in a package of claim 1, wherein the SOC further comprises a dedicated temperature sensor that can then sense the operating temperature of the gas sensor array.

33. The sensor in a package of claim 22, wherein the temperature sensor comprises internal bipolar transistors in a differential configuration.

34. The sensor in a package of claim 1, wherein the sensor chip is stacked on top of the SoC within the package, and wherein the package is a Land Grid Array.

35. The sensor in a package of claim 24, wherein the package comprises a lid, and the lid comprises a hole that to provide an air interface to the sensor chip.
